(19) United States
(12) Patent Application Publication
(10) Pub. No.: US 2005/0096622 A1
(43) Pub. Date: May 5, 2005

(54) Substantially serpentine shaped tampon with varying density regions

(75) Inventor: Nanda Christine Almond, Walton, KY (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/700,776

(22) Filed: Nov. 4, 2003

(51) Int. Cl. A61F 13/15; A61F 13/20

(52) U.S. Cl. 604/385.18; 604/904

(57) ABSTRACT

A shaped tampon with a substantially serpentine outer surface is provided. The tampon has a longitudinal centerline and a cross-sectional area defined, orthogonal to the centerline and a mass of absorbent material formed into a self-sustaining shape. The tampon has an insertion end region; a withdrawal end region; and a center region. The insertion end region has an insertion end fiber density. Also, the tampon has a withdrawal end region. The withdrawal end region is opposite to the insertion end region. The withdrawal end has a withdrawal end region fiber density. Finally, the shaped tampon has a center region which is intermediate to the insertion end region and to the withdrawal end region. The center region has a center region fiber density. The insertion end region fiber density is greater than the center region fiber density.

### FIELD OF THE INVENTION

This invention relates to shaped absorbent tampons having varying density regions along the longitudinal centerline of the tampon. The invention also relates to tightly conforming wrappers and to tampon applicators useful with the shaped absorbent tampons of the present invention.

### BACKGROUND OF THE INVENTION

A wide variety of absorbent catamenial tampons have long been known in the art. Most commercially available tampons are substantially cylindrical in shape prior to use to facilitate vaginal insertion. It is well known that the vaginal canal is not smooth and linear, but rather is very contoured. Some tampons have tapered insertion end regions to make insertion more comfortable. Others have flared withdrawal end regions, perhaps to provide a larger surface area for the user to push against during insertion. Nevertheless, the inventors of the present invention have learned that comfort and/or ease of the insertion of tampons remains an important unmet consumer need. It is also important to have a tampon which is comfortable once inside the contoured vaginal canal.

Another drawback associated with tampons is poor fluid acquisition and poor absorption capacity. Fluid acquisition is important to absorb fluid quickly to prevent bypass leakage. Therefore, it is desirable that the features rendering a tampon comfortable and/or easy to insert do not compromise fluid acquisition. Moreover, features rendering a tampon comfortable and/or easy to insert should enhance the fluid acquisition capabilities of the tampon in use.

The present invention provides comfortable shaped tampons. The shaped tampons aid in the insertion ease and/or comfort. The tampons of the present invention have varying density regions to further facilitate insertion ease and/or comfort while enhancing fluid acquisition into the tampon.

### SUMMARY OF THE INVENTION

This invention relates to shaped tampon having varying density regions. A tampon is formed into a self-sustaining shape having an outer surface, which is substantially serpentine. The tampon has a longitudinal centerline and a cross-sectional area defined, orthogonal to the centerline and a mass of absorbent material formed into a self-sustaining shape.

The tampon has an insertion end region, a withdrawal end region, and a center region. The insertion end region has an insertion end fiber density. The insertion end could be tapered or flared. Also, the tampon has a withdrawal end region. The withdrawal end region is opposite to the insertion end region. The withdrawal end has a withdrawal end region fiber density. The withdrawal end region could be flared or tapered. Alternatively, the withdrawal end could be asymmetric about the longitudinal centerline of the tampon. The withdrawal end region has a withdrawal edge located at the most distal end of the tampon along the longitudinal centerline comprising a withdrawal edge fiber density. Finally, the shaped tampon has a center region which is intermediate to the insertion end region and to the withdrawal end region. The center region comprises a center region fiber density. The insertion end region fiber density is greater than the center region fiber density. The insertion end fiber density and the withdrawal end region fiber density may be about equal. Alternatively, the withdrawal end region fiber density may be greater than the center region fiber density.

The tampon's insertion end has a maximum perimeter region which has a maximum perimeter region average fiber density. The tampon's center region has a minimum perimeter region which has a minimum perimeter region average fiber density. The maximum perimeter region average fiber density which is located in the tampon's insertion end can be greater than the minimum perimeter region average fiber density which is located in the tampon's center region. The maximum perimeter region average fiber density which is located in the tampon's insertion end can be from about 105% to about 150% of the minimum perimeter region average fiber density which is located in the tampon's center region. Also, the maximum perimeter region average fiber density which is located in the tampon's insertion end could be from about 110% to about 130% of the minimum perimeter region average fiber density which is located in the tampon's center region.

Also, the maximum perimeter region average fiber density which is located in the tampon's insertion end could be from about 110% to about 130% of the withdrawal end average density. The withdrawal end region average fiber density could be more than the minimum perimeter region average fiber density which is located in the tampon's center region. Specifically, the withdrawal end region average fiber density can be from about at least 105% to about 150% of the minimum perimeter average fiber density which is located in the tampon's center region.

Both the maximum perimeter region which is located in the tampon's insertion end and the withdrawal end region could comprise a cotton and rayon blend having a first average fiber density and the minimum perimeter region which is located in the tampon's center region could comprise a cotton and rayon blend having a second average fiber density which is less than the first average fiber density.

The tampons may also optionally utilize tampon applicators which allow the user to visualize the shape of the tampon prior to use. The tampon can be housed in an applicator and the tampon applicator is at least partially translucent, allowing at least a portion of the tampon to be visible to a user prior to use. The insertion end of the applicator could also comprise flexible material. At least a portion of the flexible material conforms to at least a portion of the substantially serpentine outer surface of the tampon, enabling a user to observe at least a portion of the substantially serpentine outer surface of the tampon through the flexible material prior to expulsion of the tampon from the applicator.

The tampons of the present invention may optionally include tightly conforming wrappers which may aid in maintaining the tampon's self-sustaining shape prior to use.

### BRIEF DESCRIPTION OF THE DRAWINGS

While the specification concludes with claims particularly pointing out and distinctly claiming the subject matter which is regarded as forming the present invention, it is believed that the invention will be better understood from the following description taken in conjunction with the accompanying drawings, in which:

FIG. 1 is a plan view of the outer surface of a shaped tampon of the present invention.

FIG. 2 is a perspective view of a shaped tampon of the present invention.

FIG. 3 is a lengthwise cross-section along the longitudinal centerline showing the substantially serpentine line and the inflection point of the present invention.

FIG. 4 is a perspective view of an alternative embodiment of the shaped tampon of the present invention.

FIG. 5 is a perspective view of another alternative embodiment of the shaped tampon of the present invention.

FIG. 6 is an end view of the embodiment shown in FIG. 5 showing multiple perimeter lines of the present invention.

### DETAILED DESCRIPTION OF THE INVENTION

The present invention is directed to a shaped tampon. The FIGS. show various embodiments of such a shaped tampon 20. The present invention, however, is not limited to a structure having the particular configurations shown in the drawings.

The term “tampon” refers to any type of absorbent structure which can be inserted into the vaginal canal or other body cavities for the absorption of fluid therefrom. The “outer surface” of a tampon refers to the visible surface of the (compressed and/or shaped) tampon prior to use and/or expansion. The outer surface may optionally be aesthetically textured, such as with longitudinal rugosities, ribs, spiraling ribs, a mesh pattern, etc. Typically, tampons are constructed from an absorbent material, which has been compressed and/or shaped, in any or all of the directions including the radial direction and the axial direction, to provide a tampon which is of a size and stability to allow insertion within the vagina or other body cavity. A tampon has a “self-sustaining shape” when a tampon pledget has been compressed and/or shaped such that it assumes a general shape and size, which is vaginally insertable, absent external forces. It will be understood by one of skill in the art that this self-sustaining shape need not, and preferably does not, persist during actual use of the tampon. That is, once the tampon is inserted and begins to acquire fluid, the tampon may begin to expand and may lose its self-sustaining form.

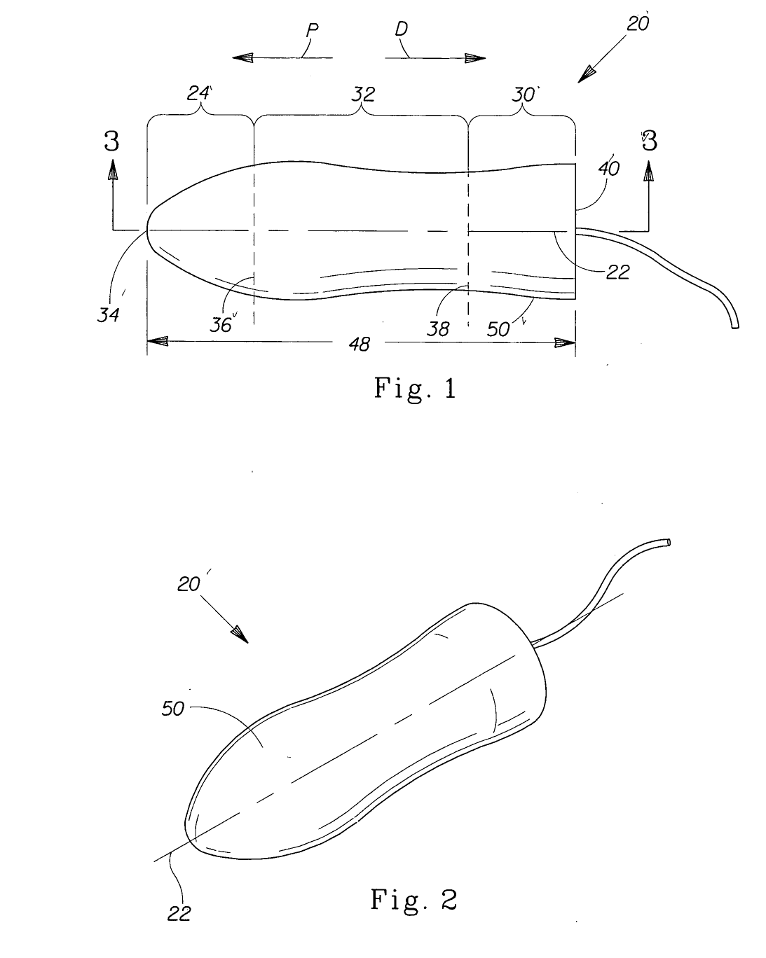

The relative positional terms “distal” and “proximal”, respectively designated P and D in FIG. 1, herein refer to directions away from and towards the body of the tampon wearer, respectively, unless otherwise specified.

As used herein, the terms “pledget” or “tampon pledget” are intended to be interchangeable and refer to a construction of absorbent material prior to the compression of such construction into a tampon as described above. Tampon pledgets are sometimes referred to as a tampon blank or a softwind, and the term “pledget” is intended to include such terms as well. In general, in this specification, the term “tampon” is used to refer to a finished tampon after the compression and/or shaping process. It will be recognized by those of skill in the art that in some contexts these terms are interchangeable. The different stages of tampon manufacture are described herein with an eye toward providing the greatest possible clarity.

As used herein, the terms “vaginal cavity,” “within the vagina,” and “vaginal canal” are intended to be synonymous and refer to the internal genitalia of the human female in the pudendal region of the body. The term “vaginal canal” is intended to refer to the space located between the introitus of the vagina (sometimes referred to as the sphincter of the vagina) and the cervix and is not intended to include the interlabial space, including the floor of vestibule. The externally visible genitalia generally are not included within the term “vaginal canal” as used herein.

The term “digital tampon” refers to a tampon which is intended to be inserted into the vaginal canal with the user's finger and without the aid of an applicator. Thus, digital tampons are typically visible to the consumer prior to use rather than being housed in an applicator.

The “longitudinal centerline” 22 of a tampon is the centerline which runs longitudinally through the center of the tampon as shown in FIG. 1. A portion of the tampon may be asymmetric about the longitudinal centerline, such as when a withdrawal end region is flared and distorted from the original shape of the rest of the tampon (such as a “fin shape”). Further, the longitudinal centerline may be linear or non-linear. The “perimeter” of a segment of the tampon is a distance measured around the outer surface of the tampon perpendicular to the longitudinal centerline. In cases where the longitudinal centerline is non-linear, the cross-sectional plane is drawn perpendicular to a line tangent to the longitudinal centerline at the point of interest. The perimeter may be measured, for instance, using Resin Embedded Microtome along with Scanning Electron Microscopy—S.E.M. (supplied by companies such as Resolution Sciences Corporation; Corte Madera, Calif.).

Tampons having “continually changing volume” are those where sequential segments of the tampon, taken along the longitudinal centerline, have different volumes. In other words, the cross-sectional area is variable along the longitudinal centerline. The cross-sectional area is orthogonal to the longitudinal centerline. For purposes of determining whether or not a tampon has constantly changing volumes herein, the volume of slices of tampons taken every 5 mm along the longitudinal centerline may be compared. Such measurements can be determined using Resin Embedded Microtome along with Scanning Electron Microscopy—S.E.M. (supplied by companies such as Resolution Sciences Corporation; Corte Madera, Calif.).

The phrase “substantially serpentine” refers to a varying cross-section between any two points on the outer surface spaced at least about 15 mm apart. The outer surface of the tampon is “substantially serpentine” when a “substantially serpentine line” is formed by the intersection of the outer surface with a plane passing through the longitudinal centerline of a tampon. In other words, if the line formed from this intersection contains no portion greater than about 15 mm long which is linear, it is said to be “substantially serpentine”.

The “inflection point” of an outer surface is the position on the outer surface that denotes a change in slope concave upward from one concave downward and vice versa.

The term “insertion edge” refers to the plane containing the absolute end of the insertion end region. In other words, the insertion edge is the most proximal end of the tampon along the longitudinal centerline. The phrase “insertion end region” refers to the end of the tampon, beginning with the insertion edge, which is intended to lead insertion of the tampon into the vagina. The insertion end region begins at the insertion edge and ends at the “first transition portion.” In one non-limiting example, this is the position which is about ¼ the total length of the self-sustaining shape down the longitudinal centerline from the insertion edge. In this non-limiting example, as a result, the total length of the insertion end region may be about ¼ the total length of the self-sustaining shape.

The term “center region” refers to the portion of the tampon located between the insertion end region and the withdrawal end region. The center region begins at the first transition portion. The center region ends at a “second transition portion.” In one non-limiting example, the second transition portion's position is about ¾ the total length of the self-sustaining shape down the longitudinal centerline from the insertion edge. In this non-limiting example, as a result, the length of the center region may be about ½ the total length of the tampon.

The term “withdrawal edge” refers to the plane containing the absolute end of the withdrawal end region. In other words, the withdrawal edge is the most distal end of the tampon along the longitudinal centerline. The withdrawal edge of the tampon does not include the length of any overwrap, secondary absorbent member, or withdrawal cord which extends beyond the main absorbent material. The phrase “withdrawal end region” refers to the region at the end of the tampon opposite the insertion end region. The withdrawal end regions begins with the second transition portion along the longitudinal centerline. The withdrawal end region terminates at the withdrawal edge. In one non-limiting example, the length of the withdrawal end region may be about ¼ of the total length of the tampon.

The term “largest insertion end region perimeter” refers to the largest perimeter within the insertion end region, excluding the perimeter at the first transition portion. The term “smallest withdrawal end region perimeter” refers to the smallest perimeter within the withdrawal end region, excluding the perimeter at the second transition portion.

The “total length of the tampon” refers to the length of the longitudinal centerline of the tampon beginning at the insertion edge and ending at the withdrawal edge. Thus, the total length of the tampon does not include the length of any overwrap, secondary absorbent member, or withdrawal cord which extends beyond the main absorbent material ending at the withdrawal edge.

The term “tapered” refers to a gradually narrowing portion of a tampon. For example, an insertion end region is “tapered” when the insertion end region or a portion thereof has a plurality of gradually decreasing perimeters approaching the insertion edge. The term “flared” refers to a widening portion of a tampon. For example, a withdrawal end region is “flared” when the withdrawal end region or a portion thereof has a plurality of gradually increasing perimeters approaching the withdrawal edge.

The “average fiber density” of a region, refers to the average fiber density of the fibers in the region. When considering the variable density of the tampon, the overwrap of the tampon, secondary absorbent member, and/or the withdrawal cord which extends beyond the main absorbent material are not included in the variable density measurements for the tampon. The average fiber density may be measured using the Test Method disclosed herein.

The phrase “maximum perimeter region” refers to a region on the tampon measuring 5 mm on either side along the longitudinal centerline 22 of the maximum perimeter of the insertion end region. Thus, the maximum perimeter region is a region that is 10 mm long. The phrase “minimum perimeter region” refers to a region on the tampon measuring 5 mm on either side along the longitudinal centerline 22 of the minimum perimeter of the center region. Thus, the minimum perimeter region is a region that is 10 mm long.

The abbreviation “g/cm²” is “grams per square centimeter”. The abbreviation “mm” is millimeter. The abbreviation mm³ is cubic millimeters.

FIG. 1 is a plan view of one embodiment of the shaped tampon 20 of the present invention, showing the relative positional terms “proximal” and “distal” designated as P and D. The tampon 20 has a total length 48 and is a mass of absorbent material compressed into a self-sustaining shape comprising a substantially serpentine outer surface 50. The tampon 20 has a longitudinal centerline 22 that runs lengthwise through the tampon 20. The tampon 20 has an insertion end region 24, a withdrawal end region 30, and a center region 32 that is located between the insertion end region 24 and the withdrawal end region 30. The insertion end region 24 begins with insertion edge 34 and ends at the first transition portion 36. In one embodiment, the insertion end region 24 is about ¼ of the self sustaining tampon 20 down the longitudinal centerline 22 from the insertion edge 34. The center region 32 begins at the first transition portion 36 and ends at the second transition portion 38. The withdrawal end region 30 is opposed to the insertion end region 24 and begins at the second transition portion 38 and terminates at the withdrawal edge 40. In one non-limiting example, the withdrawal end region 30 may be about ¼ of the total length of the tampon 20. The tampon 20 may be partially flared and partially tapered.

FIG. 2 shows a perspective view of one embodiment of the shaped tampon 20. The tampon 20 has a longitudinal centerline 22 that runs lengthwise through the tampon 20. The outer surface 50 of the tampon 20 is shown.

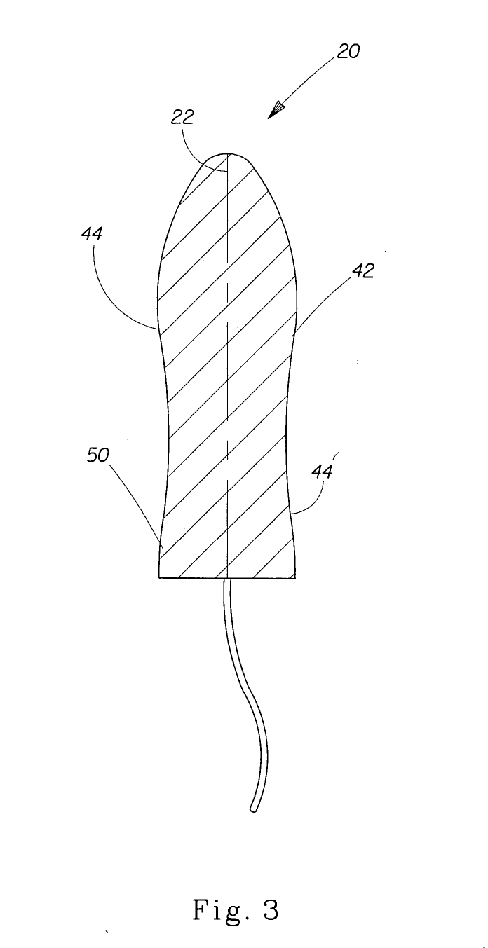

FIG. 3 is a lengthwise cross-section of the shaped tampon 20 along the longitudinal centerline 22, showing a substantially serpentine line 42 and an inflection point 44. The substantially serpentine line 42 is formed by the intersection of the outer surface 50 with a plane passing through the longitudinal centerline 22 of the tampon 20.

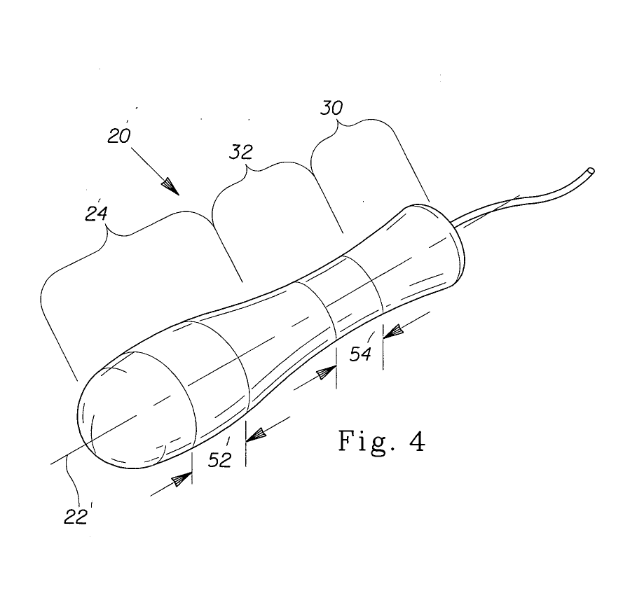

FIG. 4 shows an alternative embodiment of the shaped tampon 20 of the present invention. The tampon 20 has an insertion end region 24, a withdrawal end region 30, a center region 32 that is located between the insertion end region 24 and the withdrawal end region 30, a maximum perimeter region 52, and a minimum perimeter region 54. Both the maximum perimeter 52 region and the minimum perimeter region 54 have a dimension of about 10 mm as measured along the longitudinal centerline 22. In this embodiment, the perimeter in the center region is less than both a largest insertion end region perimeter and the smallest withdrawal end region perimeter.

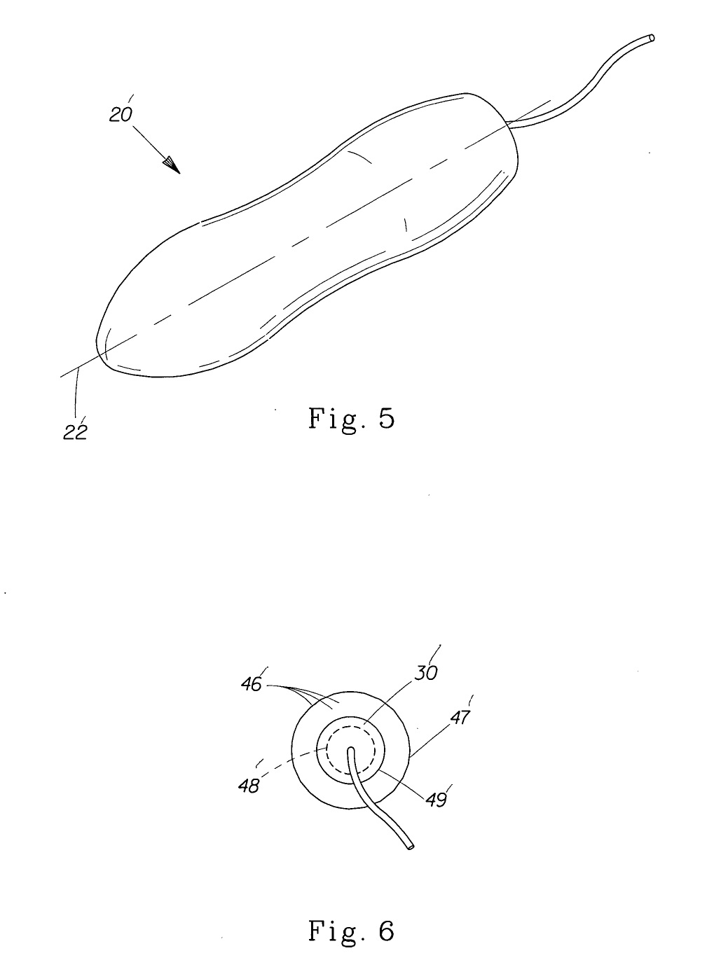

FIG. 5 is a perspective view of another embodiment of the shaped tampon 20 of the present invention. The tampon 20 has a longitudinal centerline 22 that runs lengthwise through the tampon 20. The shaped tampon 20 has variable densities along the longitudinal centerline 22.

FIG. 6 is an end view from the withdrawal end region 30 of the embodiment, showing multiple perimeter lines 46. Specifically, FIG. 6 shows the perimeter 47 of the maximum perimeter region 52 (shown in FIG. 4) located in the insertion end region 24 (shown in FIG. 4), the perimeter 48 of the minimum perimeter region 54 (shown in FIG. 4) located in center region 32 (shown in FIG. 4), and the perimeter 49 of the withdrawal end region 30.

I. Shaped Tampons of the Present Invention

In order to better understand the present invention, a detailed description of several preferred embodiments is given. This description is intended to be by way of example and not to limit the invention to these preferred embodiments. One of ordinary skill in the art will appreciate from this description how to make and use tampons incorporating the various features of the present invention, although not every conventional feature is described in undue detail. As seen in FIG. 1, the tampon 20 of the present invention comprises a mass of absorbent material compressed into a self-sustaining shape which comprises a withdrawal end region 30, a center region 32, and an insertion end region 24.

The insertion end region 24 of the tampon 20 of the present invention may be entirely or partially tapered. Alternatively, the insertion end region 24 of the tampon 20 of the present invention may be entirely or partially flared. The center region 32 of the tampon 20 of the present invention may be entirely or partially flared. Alternatively, the center region 32 of the tampon 20 of the present invention may be entirely or partially tapered. In one non-limiting example, at least a portion of the withdrawal end region 30 of a tampon 20 according to the present invention may be flared, beginning, for instance, at the second transition portion 38. In the case where the entire withdrawal end region 30 may be flared, the withdrawal edge 40 may have a larger perimeter than the second transition portion 38. Optionally, only a portion of the withdrawal end region 30 could be flared, followed by a tapered portion which terminates at the withdrawal edge 40. In this case, the withdrawal end region 30 may have a perimeter which is greater than the withdrawal edge perimeter. In other words, the perimeter increases and then decreases between the second transition portion 38 and the withdrawal edge 40. In another embodiment, at least a portion of the insertion end region 24 is tapered, terminating with the insertion edge 34 which is the smallest perimeter of the insertion end region 24.

A. Outer Surfaces which are Substantially Serpentine and Define Continually Changing Volumes

As seen in FIG. 2, the present invention relates to tampons which comprise a self-sustaining shape having an outer surface 50 which defines continually changing volumes along the longitudinal centerline 22. In other words, the cross-sectional area is variable along the longitudinal centerline 22. Further, the tampon 20 has an outer surface 50 which is substantially serpentine. For example, the continually changing volumes may comprise a series of 5 mm regions along the longitudinal centerline 22 having increasing volumes. These increasing volumes could be followed by a series of 5 mm regions having decreasing volumes. These decreasing volumes could be followed by another series of 5 mm regions having a series of increasing volumes. Alternatively, the continually changing volumes may comprise a series of 5 mm regions having decreasing volumes. These decreasing volumes could be followed by a series of 5 mm regions having increasing volumes. These increasing volumes could be followed by another series of 5 mm regions having a series of decreasing volumes. It should be understood that a tampon 20 is considered to have continually changing volumes despite the possibility that two sequential 5 mm regions have the same volume. Referring to FIG. 1, for instance, this may occur where the two sequential 5 mm regions divide the center region 32. As seen in FIG. 3, each of the above particular embodiments defines at least one inflection point 44 on the substantially serpentine line 42. Other embodiments further comprising additional series of increasing and/or decreasing volumes are also envisioned, which result in at least two inflection points 44 on the substantially serpentine line 42.

In addition to having an outer surface which defines continually changing volumes and is substantially serpentine, certain embodiments may optionally have perimeter restrictions such as those described below in section B.

B. Embodiments Comparing the Perimeters of the Center Region to the Perimeters of the Insertion End Region and the Perimeters of the Withdrawal End Region

As seen in FIG. 1, tampon 20 of the present invention may optionally also have at least one perimeter in the center region 32 which is less than the largest insertion end region perimeter and/or a withdrawal end region perimeter. As shown, the center region 32 has at least one perimeter which is less than all withdrawal end region perimeters, such as when the entire withdrawal end region 30 is flared. Thus, embodiments fitting this description may have at least one “waist” or narrowing point in the center region 32 as compared to the withdrawal end region 30 and the insertion end region 24, which can readily be seen, for example, in FIGS. 1-5.

Embodiments included within this description also include, but are not limited to tampons 20 wherein at least one of the center region perimeters is from about 5% to about 35%, alternatively, from about 15% to about 25% smaller than the largest insertion end region perimeter. Similarly, at least one of the center region perimeters may be from about 5% to about 35%, alternatively, from about 15% to about 25% smaller than the largest withdrawal end region perimeter.

C. Density Changes Along the Longitudinal Centerline

It is desirable to have fiber density changes along the longitudinal centerline 22 of the present invention. Such density changes increase comfort and fluid acquisition. Such density changes apply to all shaped tampons in addition to the shaped tampons described above. As seen in FIG. 4, in particular, it is desirable to have the average fiber density of the withdrawal end region 30 more than the average fiber density of the minimum perimeter region 54, located in the center region 32, which is often, but not necessarily located at the “waist” of the center region 32. Also, it is desirable to have the average fiber density of the insertion end region 24 more than the average fiber density of the minimum perimeter region 54, located in the center region 32. Similarly, it is desirable to have the average fiber density of the maximum perimeter region 52 located in the insertion end region 24, which typically resembles a bulb near the insertion end region 24 of the center region 54, which is more than the average fiber density of the minimum perimeter region 54 located in the center region 32.

Also, it is desirable to have the average fiber density of the withdrawal end region 30 and the insertion end region 24 more than the average fiber density of the center region 32. Additionally, it is desirable to have the average fiber density of the insertion end region 24 greater than the average fiber density of the withdrawal end region 30 and the center region 32. In another embodiment, the average fiber density of the insertion end region 24 is greater than the average fiber density of the withdrawal end region 30 and the center region 32 where the withdrawal end region 30 and the center region 54 may be generally equal. Such constructions not only increase comfort and insertion ease, but also provide an improved fluid acquisition benefit since the lower average fiber density regions can more quickly absorb fluid.

While it is envisioned that numerous tampons 20 can be constructed according to the present invention to have combinations of average fiber densities along the longitudinal centerline 22 where the insertion end region is greater than the center region, some examples include but are not limited to tampons which have the insertion end region 24 average fiber density that is from about 105% to about 150%, alternatively, from about 110% to about 130% of the center region 32 average fiber density. Alternatively, or additionally, the tampon 20 may have a withdrawal end region 30 average fiber density that is from about 105% to about 150%, alternatively, from about 110% to about 130% of the center region 32 average fiber density.

Generally, the insertion region 24 average fiber density can be from about 0.31 g/cc to about 0.60 g/cc. Moreover, the center region 32 average fiber density can be from about 0.25 g/cc to about 0.50 g/cc. Furthermore, the withdrawal end region 30 average fiber density can be from about 0.30 g/cc to about 0.50 g/cc.

Varying average fiber density regions can be achieved through a variety of approaches when dealing with the shaped tampons of the present invention. For instance, the tampon pledget may itself have a density profile tailored to provide the desired average fiber density regions upon compression and/or shaping. For example, a pledget may comprise low average fiber density rayon in an area near the edges of the web, which becomes at least a portion of the center of the web, and a higher average fiber density cotton/rayon blend in the insertion end region 24 and/or the withdrawal end region 30, such that when properly manufactured, the desired average fiber density regions result. Another approach to vary the average fiber density in the various regions of the shaped tampon is to include more fibers or decrease the amount of fibers, thereby increasing or decreasing the density.

Another approach is to start with different shaped tampon pledgets, resulting in varying amounts of starting material in different regions prior to compression. Useful shapes for pledgets when using this approach, include but are not limited to “softened rectangles” having curved sides, chevron shapes, or a shape resembling an open book.

When tampons of the present invention are created by rolling the pledget prior to compression and/or shaping, a portion of the absorbent material can be displaced from the withdrawal end region 30 to the insertion end region 24, as described in U.S. Pat. No. 6,283,952. Alternatively, a tapered compression rod used at the withdrawal end region 30 may be used to displace material towards the center region 32, regardless of the tampon construction.

The selection of which method(s) to employ to achieve the desired average fiber density variations described above will depend on variables such as the tampon construction and processing restrictions, the absorbent material, the desired final tampon absorbency, and the particulars of the desired shape. One of ordinary skill in the art will easily recognize how to employ these approaches individually or in combination to create the final desired product to provide the identified consumer benefits discussed above.

D. Tampon Materials and Components

The pledget may be constructed from a wide variety of liquid-absorbing materials commonly used in absorbent articles. Such materials include but are not limited to rayon (such as GALAXY Rayon (a tri-lobed rayon structure) available from Acordis Fibers Ltd., of Hollywall, England); SARILLE L rayon (a round fiber rayon both available from Acordis Fibers Ltd., of Hollywall, England); cotton; folded tissues; woven materials; nonwoven webs; synthetic and/or natural fibers or sheeting; comminuted wood pulp which is generally referred to as airfelt; or any combination of these materials. Additionally, superabsorbent materials, such as superabsorbent polymers or absorbent gelling materials, may be incorporated into the tampon.

The pledget can be rectangular or any other shape prior to compression and/or shaping. A more detailed description of liquid-absorbing materials and pledget shapes and dimensions can be found in co-pending patent application Ser. No. 10/039,979, filed Oct. 24, 2001, entitled “Improved Protection and Comfort Tampon”, to Agyapong et al., Docket Number 8758.

The tampon of the present invention optionally includes a withdrawal cord, a secondary absorbent member, a liquid permeable overwrap material, and/or an applicator. Withdrawal cords useful in the present invention may be made of any suitable material known in the prior art. Additionally, the tampons of the present invention may also benefit from a secondary absorbent member. U.S. Pat. No. 6,258,075 to Taylor et al. entitled “Tampon with Enhanced Leakage Protection” describes tampons having a variety of secondary absorbent members. Optional overwrap materials useful herein include rayon, cotton, bicomponent fibers, polyethylene, polypropylene, other suitable natural or synthetic fibers known in the art, and mixtures thereof. When considering the variable density of the tampon, the overwrap of the tampon, specifically the length of any overwrap, secondary absorbent member, and/or the withdrawal cord which extends beyond the main absorbent material, do not affect the variable density of the tampon.

The tampons of the present invention are typically inserted digitally. When the tampons are intended to be digitally inserted, it may be desirable to provide a finger indent at the withdrawal end region 30 of the tampon 20 to aid in insertion. A finger indent can be made using a compression rod. An example of finger indents is found in U.S. Pat. No. 6,283,952, filed May 5, 1997, entitled “Shaped Tampon”.

II. Optional Applicators Useful with the Shaped Tampons of the Present Invention

Alternatively, insertion may be aided through the use of any applicator adapted from the prior art for use with the shaped tampon. Prior art applicators of typically a “tube and plunger” type arrangement may be plastic, paper, or other suitable material. Additionally, a “compact” type applicator is also suitable. Since shaped tampons offer an additional consumer benefit of aesthetic appeal, it is often desirable to combine the shaped tampon with an applicator type which enables the user to observe at least a portion or the whole shape of the shaped tampon. Two techniques which allow the user to better notice the shape of the tampon are to either make visual observation possible through the use of translucent or even transparent applicator materials, or to provide a tampon applicator insertion end that better follows and hence better displays the profiled shape of the enclosed shaped tampon than the typical commercial tampon applicators comprising straight-walled cylindrical inserter tubes often made from molded plastic or laminated cardboard tubes. As used hereinafter, the phrase “translucent” is meant to include completely transparent materials as well as those having a lesser degree of transparency, yet allow for the user to see through the material to a degree sufficient to ascertain at least a portion of the shape of the tampon even in the absence of conforming the applicator shape to the profiled shape of the tampon. Optionally, an applicator may employ both techniques allowing the user to see the shaped tampon prior to use through a translucent applicator which also conforms to the profiled shape of the enclosed tampon.

The insertion end of the applicator may be rigid or flexible. Rigid insertion end structures could be shaped in a suitable manner (e.g., injection molding) to provide at least a degree of profiled shape observation. Alternatively, insertion ends of applicators made from flexible or pliable materials, such as films, paper, flexible wovens, or non-wovens, can also be used. Such flexible or pliable insertion ends include those which partially or fully enclose the tampon comprising a “sleeve” or a “tube” (as in U.S. Pat. Nos. 2,922,422 and 2,922,423); a “sheath” (as in U.S. Pat. Nos. 2,092,427 and 3,749,093); a “barrel” (as in U.S. Pat. No. 5,135,475); a “bag” (as in U.S. Pat. No. 3,358,686); or a “film enclosure” (as in U.S. Pat. No. 4,610,659).

III. Wrappers Useful with the Shaped Tampons of the Present Invention

The shaped tampons 20 of the present invention can optionally employ a wrapper to package the tampon to provide sanitary protection and ease in handling. The wrapper is tightly conforming to the outer surface 50 of the tampon 20 in order to visually show the consumer the tampon 20 packaged therein. Tightly conforming wrappers are particularly useful when the shaped tampons are intended to be used digitally and therefore are not housed in an applicator prior to use. The wrappers should substantially enclose each individual tampon 20 and are intended to be removed prior to insertion and use. “Tightly conforming” means that there is substantially no visually noticeable void space between the wrapper and the tampon 20. In other words, the perimeter of the tightly conforming wrapper does not exceed the perimeter of the outer surface 50 of the tampon 20 by more than about 50%, alternatively not more than about 30%. In yet another embodiment, the wrapper on average does not exceed the perimeter more than about 10% or even not more than about 5%. Since the perimeter of a tampon typically changes as a function of the length of the tampon 20, especially because the tampon is shaped as described herein, the aforementioned limits for the tight conformation of the wrapper apply to at least all substantially lengthwise portions of the outer surface of the tampon 20, and preferably to all portions of the outer surface 50 of the tampon 20.

Wrappers can be made to be tightly conforming through use of a variety of known techniques and/or materials. The wrapper material used can be any material suitable to be used for hygienically wrapping tampons. Suitable wrapper materials for use herein include flexible polymeric films, having a thickness of less than about 1 mm. Examples for wrapper materials suitable for use with the present invention are polymeric films made of polyethylene, polypropylene, polyester, polystyrene, cellophane, polyamide, polyvinyl chloride, ethylene-vinyl acetate copolymer, and the like. Polyolefin materials such as polyethylene and polypropylene, or polyvinyl chloride are particularly useful as heat shrinkable materials and can be so used by one of ordinary skill in the art to form tightly conforming wrappers, typically utilizing heat seals to close off the wrapper edges.

Generally, the wrapper of the present invention, in its most generic form, can be made by wrapping wrapper material around the tampon and sealing it onto itself for closing the wrapper material in order to substantially enclose the tampon. The sealing may be facilitated by pressure and optionally heat. In another embodiment of the invention, a sleeve of the wrapper film material is formed and connected with an adhesive in an overlapping region. The sleeve can be put over the tampon and then heat shrunk. If needed, the end of the wrapper being assigned to the withdrawal and/or the insertion end region of the tampon could be closed with an adhesive in order to form a pouch that is heat shrunk in the next step. When heat-shrinkable material is used, it can be shrunk after being closed around the tampon, decreasing the dimensions of the wrapper material so that the wrapper tightly conforms to the outer surface of the tampon. “Heat-shrinkable,” as used herein, refers to materials which have an extension typically in at least two dimensions, e.g., films or nonwovens, and which reduce their extension in at least one of the dimensions when being heated to an elevated temperature above normal storage or usage temperatures, but being lower than their melting temperature or being lower than their decomposition temperature in case the material decomposes prior to melting. The same can be achieved by using stretch film or even a pre-stretched elastic material, which is allowed to relax into a non- or low-tensed or non- or low-stretched state after being closed around the tampon. Another alternative for achieving a tightly conforming wrapper is partially closing the wrapper after having wrapped the wrapper material around the tampon, then evacuating the interior of the wrapper by application of vacuum, and finally, completely closing the wrapper.

IV. Making the Tampon of the Present Invention

A method useful in making the tampons of the present invention involves the following steps: providing a first split cavity mold member having a first inner surface and a first outer surface; providing a second split cavity mold member having a second inner surface and a second outer surface; facing the first inner surface of the first split cavity mold member towards the second inner surface of the second split cavity mold member which results in a split cavity mold having a first end, a second end, and an opening located in the second end; providing an outer sleeve wherein the outer sleeve has a first end, a second end, and an opening located in the second end; inserting the first end of the split cavity mold into the outer sleeve, wherein the opening in the split cavity mold is visible through the second end of the outer sleeve and the outer sleeve holds together the first split cavity mold member and the second split cavity mold member and wherein the combination of the split cavity mold and the outer sleeve forms a joined sleeve cavity mold with a transfer end; loading the joined sleeve cavity mold into a v-block holder of a tampon compression machine with the transfer end of the joined sleeve cavity mold facing a compression jaw in the tampon compression machine; providing a tampon pledget; placing the tampon pledget into the compression jaw; actuating the compression jaw thereby compressing the tampon pledget into a high aspect ratio shape resulting in a compressed tampon pledget; transferring the compressed tampon pledget from an actuated jaw into the transfer end of the joined sleeve cavity mold using a compression member resulting in a tampon mold having a first end and a second end wherein the second end has an opening; removing the compression member from the opening of the tampon mold which contains the resulting shaped tampon; removing the tampon mold which contains the shaped tampon from the tampon compression machine; self-sustaining the shaped tampon; and removing the shaped tampon from the tampon mold.

If microwaving is used as a self-sustaining method, the tampon mold is placed in a microwaving unit. The mold and the sleeve (if used) are made from a microwave-transparent material(s). After the tampon is self-sustained, the shaped tampon may be removed by removing the tampon mold from the microwaving unit. Then, the split cavity mold may be ejected from the outer sleeve through the second end of the outer sleeve. Next, the split cavity mold is split, that is at least partially separated or separated to the desired degree (e.g., partially opened) to aid the next step of tampon removal. Finally, the shaped tampon is removed from the split cavity mold.

It will be recognized by those of skill in the art that compression to a self sustaining form requires imparting both heat and pressure to the tampon pledget. Such heat and pressure cause the fibers to “set” and achieve this self-sustaining form. Details of the above described method as well as other methods which may be used to form the tampons of the present invention can be found in co-pending patent application Ser. No. 60/365,669, filed Mar. 18, 2002 entitled “Method of Producing a Shaped Tampon”, to “Sageser, et al.”.

### EXAMPLES

The following is a listing of examples illustrating various embodiments of the present invention. It would be obvious to those skilled in the art that various other changes and modifications can be made without departing from the spirit and scope of the invention.

Examples 1-9, provided below, are tampons of the present invention which have an outer surface with a substantially serpentine shape. The below tampons include tampons designed to have an absorbent capacity, as measured by the standard syngyna test, of between about 9 to about 12 grams. One of ordinary skill in the art will readily recognize that other absorbencies and sizes can be scaled up or down as desired. The total length of the tampons in the examples is 48 mm. The “Tampon Length” column below indicates the positions of the measurements on the total length of the tampon including the insertion edge and the withdrawal edge. The perimeter measurements of the tampon were taken relative to the total length of the tampon. The perimeter measurement at 0 mm is the withdrawal edge, the perimeter measurement at 48 mm is the insertion edge, other measurements listed in between fall from the withdrawal edge to the insertion edge sequentially along the longitudinal centerline.

| Tampon | Example 1 | Example 2 | Example 3 | Example 4 | Example 5 | Example 6 | | Example 8 | Example 9 |
|---|---|---|---|---|---|---|---|---|---|
| length | Perimeter | Perimeter | Perimeter | Perimeter | Perimeter | Perimeter | Example 7 | Perimeter | Perimeter |
| [mm] | [mm] | [mm] | [mm] | [mm] | [mm] | [mm] | Perimeter [mm] | [mm] | [mm] |
| 0 | 39.3 | 39.3 | 39.3 | 40.8 | 40.8 | 39.3 | 43.3 | 43.3 | 43.3 |
| 5 | 37.7 | 39.6 | 38.6 | 40.5 | 40.5 | 39.9 | 45.8 | 45.8 | 45.8 |
| 10 | 36.8 | 39.9 | 37.7 | 39.6 | 39.9 | 40.8 | 44.6 | 44.6 | 44.6 |
| 12 | 36.1 | 40.8 | 38.6 | 39.3 | 39.3 | 39.3 | 43.9 | 43.9 | 43.9 |
| 15 | 35.2 | 44.0 | 39.6 | 38.6 | 39.9 | 39.9 | 42.7 | 41.4 | 42.7 |
| 20 | 34.6 | 45.6 | 40.8 | 38.0 | 40.8 | 40.8 | 41.4 | 41.4 | 41.4 |
| 24 | 35.2 | 46.2 | 39.6 | 37.9 | 39.3 | 39.3 | 42.1 | 41.4 | 43.0 |
| 25 | 35.8 | 47.1 | 39.3 | 37.7 | 39.3 | 39.3 | 43.0 | 41.4 | 45.5 |
| 30 | 37.0 | 46.2 | 37.7 | 39.9 | 37.7 | 37.7 | 44.9 | 43.0 | 45.5 |
| 35 | 37.7 | 45.6 | 39.0 | 40.8 | 38.6 | 38.6 | 47.1 | 44.9 | 45.5 |
| 36 | 38.0 | 42.4 | 39.3 | 40.6 | 39.3 | 39.3 | 47.4 | 47.4 | 45.5 |
| 40 | 40.8 | 37.7 | 40.8 | 34.6 | 31.4 | 22.0 | 44.6 | 44.6 | 45.5 |
| 45 | 34.6 | 22.0 | 41.4 | 15.7 | 15.7 | 22.0 | 31.1 | 31.1 | 31.1 |
| 48 | 0 | 0 | 0 | 0 | 0 | 0 | 0 | 0 | 0 |

Example 10: A tampon is formed having a smallest withdrawal end region perimeter of 39.3 mm, a largest insertion end region perimeter of 40.8 mm, and a center region perimeter of less than 39.3 mm, for instance, 37.9 mm.

Example 11: A tampon is formed having at least one center region perimeter of 37.7 mm and a largest insertion end region perimeter of 40.8 mm. Thus, the center region has a perimeter which is 7.60% less than the largest insertion end region perimeter.

Example 12: A tampon according to Example 4 is made wherein the minimum perimeter of the center region is located 12 mm from the withdrawal edge along the longitudinal centerline and has a minimum perimeter of 39.3 mm and an average fiber density of 0.03960 g/cc. The maximum perimeter of the insertion end region is located 35 mm from the withdrawal edge along the longitudinal centerline and has a maximum perimeter of 40.8 mm and an average fiber density of 0.04650 g/cc. Thus, the average fiber density of the maximum perimeter region located in the insertion end region is 117% of the average fiber density of the minimum perimeter region located in the center region.

Example 13: A tampon resembling FIG. 4 is made from a chevron shaped pledget wherein the volume of 5 mm sections, beginning at the withdrawal end region and ending at the insertion end region, are as follows: 683.70 mm³, 603.50 mm³, 452.64 mm³, 452.62 mm³, 905.25 mm³, 792.40 mm³, 792.60 mm³, 141.37 mm³, 34.34 mm³.

Examples 14-15, provided below, are tampons of the present invention which have an outer surface with a substantially serpentine shape. The below tampons are designed to have an absorbent capacity as measured by the standard syngyna test, of between about 9 to about 12 grams. One of ordinary skill in the art will readily recognize that other absorbencies and sizes can be scaled up or down as desired. The total length of the tampons in the examples is 48 mm.

The substantially serpentine shaped tampon densities are measured along three regions: the insertion end region, the center region, and the withdrawal end region.

| | Density in the | Density in | Density in the |
|---|---|---|---|
| | Insertion End | the Center | Withdrawal End |
| | Region | Region | Region |
| Example | [g/cc] | [g/cc] | [g/cc] |
| 14 | .35 | .50 | .73 |
| 15 | .40 | .45 | .60 |
| 16 | .36 | .40 | .58 |
| 17 | ,34 | .31 | .34 |

Examples 18-21, provided below, are tampons of the present invention which have an outer surface with a substantially serpentine shape. The below tampons are designed to have an absorbent capacity as measured by the standard syngyna test, of between about 9 to about 12 grams. One of ordinary skill in the art will readily recognize that other absorbencies and sizes can be scaled up or down as desired. The total length of the tampons in the examples is 48 mm.

The shaped tampon densities are measured per the regions below.

| | Density at the | | |
|---|---|---|---|
| | Maximum | Density at the | |
| | Perimeter in the | Minimum | Density in the |
| | Insertion End | Perimeter in the | Withdrawal End |
| | Region | Center Region | Region |
| Example | [g/cc] | [g/cc] | [g/cc] |
| 18 | .42 | .47 | .63 |
| 19 | .42 | .42 | .57 |
| 20 | .39 | .42 | .45 |
| 21 | ,44 | .33 | .38 |

Density Test Method

The density test method is conducted to determine the density of the absorbent article, specifically, digital tampons. The test procedure determines the density profile of a digital tampon using a cross-sectional method.

The equipment and materials needed for the test are as follows:
1. Razor (X-ACTO)
2. Mass balance Mettler PM400 (Mettler Instrument Corp., Highstown, N.J.) accurate to 0.001 g
3. Mass balance adventurer (OHAUS Corp., Pine Brook, N.J.) accurate to 0.001 g
4. Stopwatch, readable to 1.0 second (West Chester, Pa.)
5. ACS grade Isopropanol (West Chester, Pa.)

The samples to be tested should be conditioned as follows:
1. Each sample is held at room temperature of 74° F.±1° F. and at a relative humidity of 50%±2% for 2 hours.
2. Cut the cord and the tail.
3. Discard the cord and the tail.
4. Each tampon is laid on a flat surface.
5. Each tampon is cut into three sections using a razor blade/Xacto knife. The cuts should not disturb the cross-sectional profile. The following are the steps for cutting the tampon: 1) cut through the surface of the tampon; 2) turn the tampon and cut through surface; 3) identify the compression direction during cutting the surface; and
   4) cut through at the compressed direction to minimize any disturbance of the tampon structure. Discard any tampons if there any signs of structure change.
   a. First section: The tampon is cut 12 mm from the tip. Thus, the total length of the first section is 12 mm. This section is considered the insertion end.
   b. Second Section: The tampon is cut 24 mm from the end. This section is considered the center region. Thus, the total length of the second section is 24 mm.
   c. Third Section: A third piece is left. This section is considered the withdrawal end region.
6. Weigh the first section (W1). This weight is designated as W₁.
7. Record this measurement. This measurement represents the first section's mass in grams.
8. Weigh the second section (W2). This weight is designated as W₂.
9. Record this measurement. This measurement represents the second section's mass in grams.
10. Weigh the third section (W3). This weight is designated as W₃.
11. Record this measurement. This measurement represents the third section's mass in grams.
12. Calculate the total weight of the tampon designated as Wₜ using the formula Wₜ=W₁+W₂+W₃.
13. Place ACS grade Isopropanol in a cylinder.
14. Put the insertion section into a Teflon tube with the ring at one end.
15. Dip the Teflon tube with the insertion section into excess amount of ACS grade Isopropanol for 30 seconds.
16. Remove the Teflon tube with the insertion section from the Isopropanol and drain for 30 seconds.
17. Dip the Teflon tube with the insertion section into another Teflon tube filled with Isopropanol and the amount of isopropanol displaced is collected and weighed using a calibrated mass balance adventurer (OHAUS corp, NJ) accurate to 0.001 g.
18. The volume of the insertion section is calculated from the weight of displaced amount of Isopropanol as V1=Wᵢₛₒₚᵣₒₚₐₙₒ/0.78 (density of Isopropanol) (cc).
19. Determine the density in g/cc of the first section by dividing the mass of the first section by the final volume number.
20. Repeat steps 11-17 to determine the volume of second section (V2) and whole tampon (Wh). Determine the density of the second section (D2) and the whole tampon (Dh).
21. Take an uncut tampon and weigh the whole tampon (Wh)
22. Record this measurement. This measurement is denoted and recorded and represents the whole tampon's mass in grams.
23. Repeat step 11-17 to determine the density of the whole tampon (Dh).
24. The density of the third section was calculated as follows:
   D3=W₃/V3=W₃/(Wt/Dh−V1−V2).

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A tampon comprising a longitudinal centerline and a cross-sectional area defined, orthogonal to said centerline and a mass of absorbent material formed into a self-sustaining shape, said self-sustaining tampon comprising:
a.) an insertion end region comprising an insertion end fiber density;
b.) a withdrawal end region opposite said insertion end region, wherein said withdrawal end region comprises a withdrawal end region fiber density; and
c.) a center region intermediate said insertion end region and said withdrawal end region, wherein said center region comprises a center region fiber density;
wherein said self-sustaining shape has an outer surface which is substantially serpentine;
and wherein said insertion end region fiber density is greater than said center region fiber density.

2. The tampon according to claim 1 wherein said insertion end region is tapered and said withdrawal end region is flared.

3. The tampon according to claim 1 wherein said insertion end region is flared and said withdrawal end region is tapered.

4. The tampon according to claim 1 wherein said insertion end region is tapered and said withdrawal end region is tapered.

5. The tampon according to claim 1 wherein said insertion end region is tapered and flared and said withdrawal end region is tapered and flared.

6. The tampon according to claim 1 wherein said withdrawal end is asymmetric about said longitudinal centerline of said tampon.

7. The tampon according to claim 1 wherein said insertion end fiber density and said withdrawal region fiber density are about equal.

8. The tampon according to claim 1 wherein said insertion end has a maximum perimeter region in said insertion end region and a maximum perimeter region average fiber density, and wherein said center region has a minimum perimeter region and a minimum perimeter region average fiber density;
wherein said maximum perimeter region average fiber density is greater than said minimum perimeter region average fiber density.

9. The tampon according to claim 8 wherein said maximum perimeter region average fiber density located in said insertion end region is from about 105% to about 150% of said minimum perimeter region average fiber density located in said center region.

10. The tampon according to claim 8 wherein said maximum perimeter region average fiber density located in said insertion end region is from about 110% to about 130% of said minimum perimeter region average fiber density located in said center region.

11. The tampon according to claim 8 wherein said maximum perimeter region comprises a cotton and rayon blend having a first average fiber density and said minimum perimeter region comprises a cotton and rayon blend having a second average fiber density which is less than said first average fiber density.

12. The tampon according to claim 1 further comprising an applicator wherein said tampon is housed in said applicator.

13. The tampon according to claim 1 further comprising an applicator wherein said applicator is at least partially translucent, allowing at least a portion of said tampon to be visible to a user prior to use.

14. The tampon according to claim 1 further comprising an applicator wherein said tampon is housed in said applicator; wherein said applicator comprises an insertion end region comprising flexible material;
and wherein at least a portion of said flexible material conforms to at least a portion of said substantially serpentine outer surface of said tampon, enabling a user to observe at least a portion of said substantially serpentine outer surface of said tampon through said flexible material prior to expulsion of said tampon from said applicator.

15. The tampon according to claim 1 further comprising a wrapper wherein said wrapper is tightly conforming.

16. The tampon according to claim 1 further comprising texturing on said outer surface of said tampon.
